(19) United States
(12) Patent Application Publication
(10) Pub. No.: US 2013/0022495 A1
(43) Pub. Date: Jan. 24, 2013

(54) Sterilization Techniques for Surgical Instruments

(71) Applicants: James D. Allen, IV, Broomfield, CO (US); Robert M. Sharp, Boulder, CO (US); Glenn A. Horner, Boulder, CO (US); Duane E. Kerr, Loveland, CO (US); Arlan J. Reschke, Longmont, CO (US); Robert B. Stoddard, Steamboat Springs, CO (US)

(72) Inventors: James D. Allen, IV, Broomfield, CO (US); Robert M. Sharp, Boulder, CO (US); Glenn A. Horner, Boulder, CO (US); Duane E. Kerr, Loveland, CO (US); Arlan J. Reschke, Longmont, CO (US); Robert B. Stoddard, Steamboat Springs, CO (US)

(73) Assignee: TYCO Healthcare Group LP, Boulder, CO (US)

(21) Appl. No.: 13/185,593

(22) Filed: Jul. 19, 2011

(51) Int. Cl. A61L 2/10; B65D 85/00; A61L 2/08; B01J 19/08; A61L 2/03

(52) U.S. Cl. 422/24; 422/186.05; 422/22; 250/492.1; 206/363

(57) ABSTRACT

A surgical system includes a surgical instrument, a generator, and a sterilization case. The surgical instrument includes an end effector assembly adapted to connect to a source of energy. The generator is configured to couple to the surgical instrument to supply energy to the end effector assembly of the surgical instrument. The sterilization case is configured to retain the surgical instrument therein and is configured to couple to the generator. The generator is further configured to supply energy to the sterilization case when coupled thereto to sterilize the surgical instrument disposed therein.

### BACKGROUND

The present disclosure relates to surgical instruments and, more particularly, to the sterilization of surgical instruments for reuse.

### TECHNICAL FIELD

A forceps is a plier-like instrument which relies on mechanical action between its jaws to grasp, clamp and constrict vessels or tissue. Electrosurgical forceps utilize both mechanical clamping action and electrical energy to affect hemostasis by heating tissue and blood vessels to coagulate and/or cauterize tissue. Certain surgical procedures require more than simply cauterizing tissue and rely on the unique combination of clamping pressure, precise electrosurgical energy control and gap distance (i.e., distance between opposing jaw members when closed about tissue) to “seal” tissue, vessels and certain vascular bundles. Typically, once a vessel is sealed, the surgeon has to accurately sever the vessel along the newly formed tissue seal. Accordingly, many vessel sealing instruments have been designed which incorporate a knife or blade member which effectively severs the tissue after forming a tissue seal,

Generally, surgical instruments, including forceps, can be classified as single-use instruments, e.g., instruments that are discarded after a single use, partially-reusable instruments, e.g., instruments including both disposable portions and portions that are sterilizable for reuse, and completely reusable instruments, e.g., instruments that are completely sterilizable for repeated use. As can be appreciated, those instruments (or components of instruments) that can be sterilized and reused help reduce the costs associated with the particular surgical procedure for which they are used. However, although reusable surgical instruments are cost-effective, it is important that these instruments (or the reusable components thereof) be efficiently and satisfactorily sterilizable for reuse.

### SUMMARY

In accordance with one embodiment of the present disclosure, a surgical system including a surgical instrument, a generator, and a sterilization case is provided. The surgical instrument includes an end effector assembly adapted to connect to a source of energy. The generator is configured to couple to the surgical instrument to supply energy to the end effector assembly of the surgical instrument. The sterilization case is configured to retain the surgical instrument therein and is configured to couple to the generator. The generator is further configured to supply energy to the sterilization case when coupled thereto to sterilize the surgical instrument disposed therein.

In one embodiment, the sterilization case includes one or more fixtures disposed therein for securing the surgical instrument in position therein.

In another embodiment, the generator is configured to supply energy to the sterilization case to create a corona about the surgical instrument to sterilize the surgical instrument via corona discharge.

In another embodiment, an electrosurgical cable is provided, the electrosurgical cable configured to couple the generator to the surgical instrument. The electrosurgical cable may further be configured to couple the generator to the sterilization case for sterilizing the surgical instrument. Alternatively, the sterilization case may be configured to couple directly to the generator.

In still another embodiment, the sterilization case includes instructions disposed on a surface thereof, e.g., for providing particular sterilization procedures and/or instructions.

In yet another embodiment, the generator is configured to supply UV energy to the sterilization case to sterilize the surgical instrument. In such an embodiment, the surgical instrument is formed from transparent components, or materials, to facilitate UV sterilization thereof.

In still yet another embodiment, the generator is configured to supply gamma ray energy to the sterilization case to sterilize the surgical instrument.

A method of surgery is also provided in accordance with the present disclosure. The method includes providing a surgical instrument including an end effector assembly, a generator, and a sterilization case. The method further includes coupling the surgical instrument to the generator, performing a surgical task wherein energy is supplied from the generator to the end effector assembly of the surgical instrument, decoupling the surgical instrument from the generator, positioning the surgical instrument within the sterilization case, coupling the sterilization case to the generator, and operating the generator to sterilize the surgical instrument disposed within the sterilization case.

In one embodiment, the generator is configured to supply energy to the sterilization case to create a corona about the surgical instrument to sterilize the surgical instrument via corona discharge.

In another embodiment, the sterilization case includes one or more fixtures disposed therein for securing the surgical instrument in position therein.

In yet another embodiment, the surgical instrument is separated the surgical instrument into multiple components prior to positioning the surgical instrument in the sterilization case to facilitate sterilization of the surgical instrument.

In still another embodiment, the generator is configured to supply UV energy to the sterilization case to sterilize the surgical instrument. The generator may alternatively or additionally be configured to supply gamma ray energy to the sterilization case to sterilize the surgical instrument.

In accordance with another aspect of the present disclosure, a sterilization case for a surgical instrument configured to be disassembled into a plurality of sterilizable components is provided. The sterilization case includes a base configured to receive each of the components of the surgical instrument therein and cover coupled to the base and moveable relative thereto between an open position permitting insertion and removal of the surgical instrument and a closed position wherein the surgical instrument is enclosed within the sterilization case. The sterilization case further includes a plurality of fixtures disposed on the base and configured to releasably secure each of the sterilizable components of the surgical instrument to the base. More specifically, each of the fixtures is designated for releasably securing a particular component of the surgical instrument to the base such that all of and only the sterilizable components of the surgical instrument are securable within the base for sterilization thereof.

In one embodiment, a marking associated with each of the fixtures is provided, i.e., a marking is positioned about or adjacent to each of the fixtures. Each marking indicates to the user which sterilizable component of the surgical instrument is to be secured to the fixture associated therewith.

In another embodiment, both the base and cover include a complementary latching component configured to releasably latch the base and cover in the closed position.

In still another embodiment, one or more of the fixtures includes a plurality of apertures defined therethrough to provide access to a portion of the surgical instrument disposed in close proximity to the fixture to facilitate sterilization of that portion of the surgical instrument.

In yet another embodiment, both the base and cover includes a plurality of apertures defined therethrough to facilitate sterilization of the surgical instrument disposed therein.

### BRIEF DESCRIPTION OF THE DRAWINGS

Various embodiments of the subject instrument are described herein with reference to the drawings wherein:

FIG. 1 is side, perspective view of a surgical system including a forceps and an electrosurgical generator in accordance with the present disclosure;

FIG. 2 is a top, perspective view of one embodiment of a sterilization case configured for use with the surgical system of FIG. 1;

FIG. 3 is a top, perspective view of the sterilization case of FIG. 2 with the top cover removed and including the surgical instrument of FIG. 1 disposed therein;

FIG. 4 is a side, perspective view of the surgical system of FIG. 1 shown including a sterilization case coupled to the generator of the surgical system for sterilization of the surgical instrument disposed within the sterilization case; and

FIG. 5 is a side, perspective view of another embodiment of a sterilization case provided in accordance with the present disclosure.

### DETAILED DESCRIPTION

Embodiments of the present disclosure are described in detail with reference to the drawing figures wherein like reference numerals identify similar or identical elements. As used herein, the term “distal” refers to the portion that is being described which is further from a user, while the term “proximal” refers to the portion that is being described which is closer to a user.

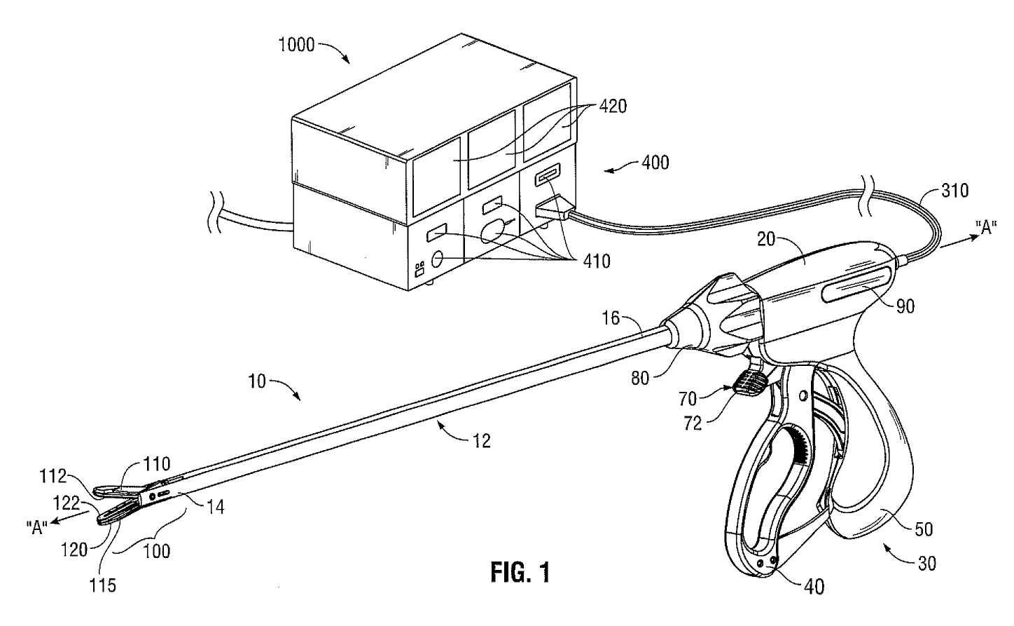

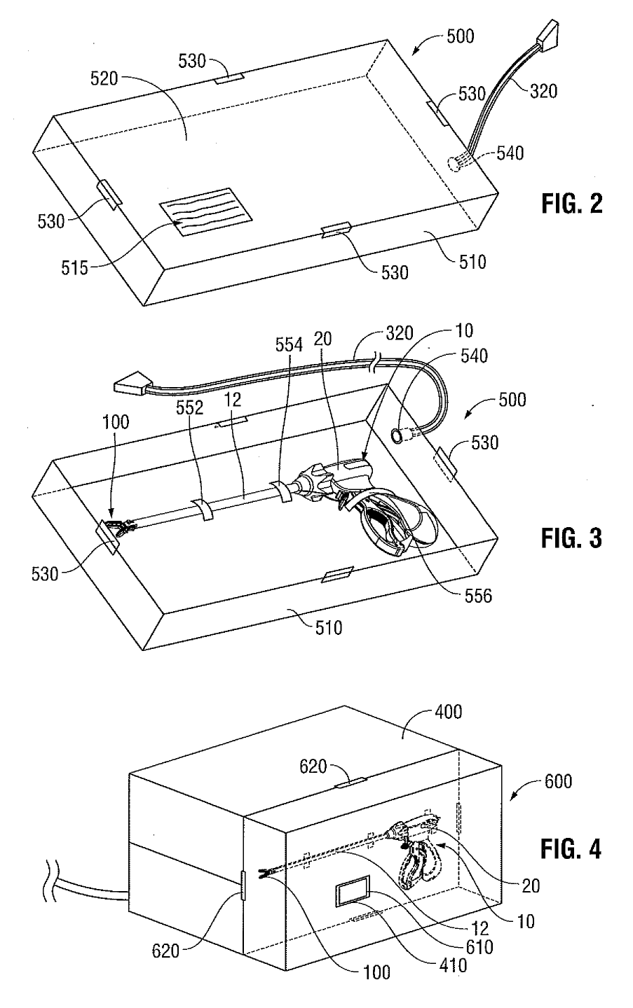

Referring now to FIG. 1, surgical system 1000 is shown including a surgical instrument, e.g., forceps 10, a surgical generator 400, and a sterilization case 500 (FIGS. 2-3). The surgical instrument may be, for example, a forceps 10. Forceps 10 is configured for use in connection with endoscopic surgical procedures, although forceps 10 may also be configured for use in connection with traditional open surgical procedures. Further, although described with reference to forceps 10 for exemplary purposes, the principles and novel features of the present disclosure apply similarly to any suitable surgical instrument, e.g., electrosurgical instruments, ultrasonic instruments, or other energy-based instruments.

Forceps 10 defines a longitudinal axis “A-A” and includes a housing 20, a handle assembly 30, a trigger assembly 70, a rotating assembly 80 and an end effector assembly 100. End effector assembly 100 includes first and second jaw members 110, 120, respectively, configured to pivot relative to one another between a spaced-apart position and an approximated position for grasping tissue therebetween. Forceps 10 further includes a shaft 12 having a distal end 14 configured to mechanically engage end effector assembly 100 and a proximal end 16 that mechanically engages housing 20.

Forceps 10 also includes an electrosurgical cable 310 that connects forceps 10 to generator 400 or other suitable power source. Cable 310 electrically connects to a wire (or wires) (not explicitly shown) disposed within housing 20 that extend through shaft 12 to ultimately connect the source of electrosurgical energy, e.g., generator 400, to jaw member 110 and/or jaw member 120 of end effector assembly 100. However, any other suitable electrical connection(s) for supplying energy to jaw member 110 and/or jaw member 120 may also be provided. Further, cable 310 may be removably engagable with housing 20, as will be described in greater detail below.

With continued reference to FIG. 1, handle assembly 30 includes a fixed handle 50 and a moveable handle 40. Fixed handle 50 is integrally associated with housing 20 and handle 40 is moveable relative to fixed handle 50. Rotating assembly 80 is rotatable in either direction about a longitudinal axis “A-A” to rotate end effector 100 about longitudinal axis “A-A.” The housing 20 houses the internal working components of the forceps 10.

End effector assembly 100, as mentioned above, is attached at a distal end 14 of shaft 12 and includes a pair of opposing jaw members 110 and 120. End effector assembly 100 is designed as a bilateral assembly, i.e., where both jaw member 110 and jaw member 120 are moveable relative to one another and with respect to shaft 12, although end effector assembly 100 may alternatively be configured as a unilateral assembly, i.e., where jaw member 120 is fixed relative to shaft 12 and jaw member 110 is moveable relative to both shaft 12 and fixed jaw member 120.

As shown in FIG. 1, each jaw member 110, 120 includes an electrically conductive tissue sealing plate 112, 122 disposed thereon. Tissue sealing plates 112, 122 are positioned on jaw members 110, 120, respectively, to define opposed tissue sealing surfaces for grasping and sealing tissue between jaw members 110, 120. In some embodiments, a knife assembly (not shown) is disposed within shaft 12 and a knife channel 115 is defined within one or both of tissue sealing plates 112, 122, of jaw members 110, 120, respectively, to permit reciprocation of a knife blade (not shown) therethrough for cutting tissue grasped between jaw members 110, 120. In such an embodiment, trigger 72 of trigger assembly 70 is operable to advance the knife blade (not shown) between a retracted position and an extended position to cut tissue grasped between jaw members 110, 120.

Continuing with reference to FIG. 1, moveable handle 40 of handle assembly 30 is ultimately connected to a drive assembly (not shown) that, together, mechanically cooperate to impart movement of jaw members 110 and 120 between a spaced-apart position and an approximated position to grasp tissue between sealing plates 112 and 122 of jaw members 110, 120, respectively. As shown in FIG. 1, moveable handle 40 is initially spaced-apart from fixed handle 50 and, correspondingly, jaw members 110, 120 are disposed in the spaced-apart position. Moveable handle 40 is depressible from this initial position to a depressed position corresponding to the approximated position of jaw members 110, 120.

Referring still to FIG. 1, electrosurgical generator 400 may be any generator suitable for use with electrosurgical devices, e.g., forceps 10, and may be configured to provide various frequencies of electromagnetic energy, e.g., radio frequency (RF), microwave, ultraviolet (UV), gamma ray, etc. More specifically, electrosurgical generator 400 includes one or more inputs 410 for coupling electrosurgical generator 400 to a surgical instrument, e.g., forceps 10, or sterilization case 500 (FIGS. 2-3), and one or more user-interface panels 420 that allow the user to select a particular mode of operation, the parameters thereof and to monitor electrical characteristics of the surgical instrument and/or tissue during use. Examples of other electrosurgical generators that may be suitable for use as a source of electrosurgical energy are commercially available under the trademarks FORCE EZ™, FORCE FX™, SURGISTAT™ II, and FORCE TRIAD™, all of which are available from Covidien Energy-Based Devices of Boulder, Colo.

In use, electrosurgical cable 310 is coupled to a desired input 410 of generator 400 at one end thereof and to housing 20 of forceps 10 at the other end thereof. Thereafter, forceps 10 is manipulated into position such that end effector assembly 100 is disposed adjacent a surgical site with tissue to be grasped, sealed and/or divided disposed between jaw members 110, 120 thereof. With forceps 10 disposed in this position, the user may depress (squeeze) moveable handle 40 from the initial position to the depressed such that jaw members 110, 120 are moved to the approximated to grasp tissue therebetween.

With tissue grasped between jaw members 110, 120, electrosurgical energy may be selectively supplied from generator 400 to end effector assembly 100, e.g., via actuation of activation switch 90. More specifically, energy is conducted between sealing plates 112, 122 of jaw members 110, 120, respectively, and through tissue to effect a tissue seal. As mentioned above, once tissue has been sealed (or simply to cut tissue, in embodiments where tissue sealing is not desired), trigger 72 of trigger assembly 70 may be actuated to advance the knife blade (not shown) between jaw members 110, 120 to cut tissue grasped therebetween. At the completion of the particular surgical task to be performed, e.g., tissue sealing and/or division, jaw members 110, 120 are returned to the spaced-apart position, e.g., by releasing, or returning moveable handle 40, and forceps 10 is withdrawn from the surgical site. As will be described in detail below, forceps 10 may then be sterilized in preparation for re-use.

With reference now to FIGS. 2-3, sterilization case 500 is shown. Sterilization case 500 may be formed from any suitable electrically-insulative material, or may be coated with an insulative material. Sterilization case 500 includes a base 510 and a removable cover 520. Base 510 and removable cover 520 each include complementary latching features 530 that are releasably engagable with one another to secure cover 520 about base 510, as shown in FIG. 2. Sterilization case 500 further includes one or more inputs 540 configured to couple sterilization case 500 to electrosurgical generator 400, e.g., via electrosurgical cable 320, electrosurgical cable 310 (FIG. 1), or any other suitable electrosurgical cable (not explicitly shown), or to couple sterilization case 500 to any other suitable sterilization equipment (not shown). Further, instructions 515 for sterilizing particular types of surgical instruments or for performing specific sterilization techniques may be provided on an outer surface of cover 520. Instructions 515 may be formed directly on cover 520, may be adhered thereto, or may otherwise be disposed thereon.

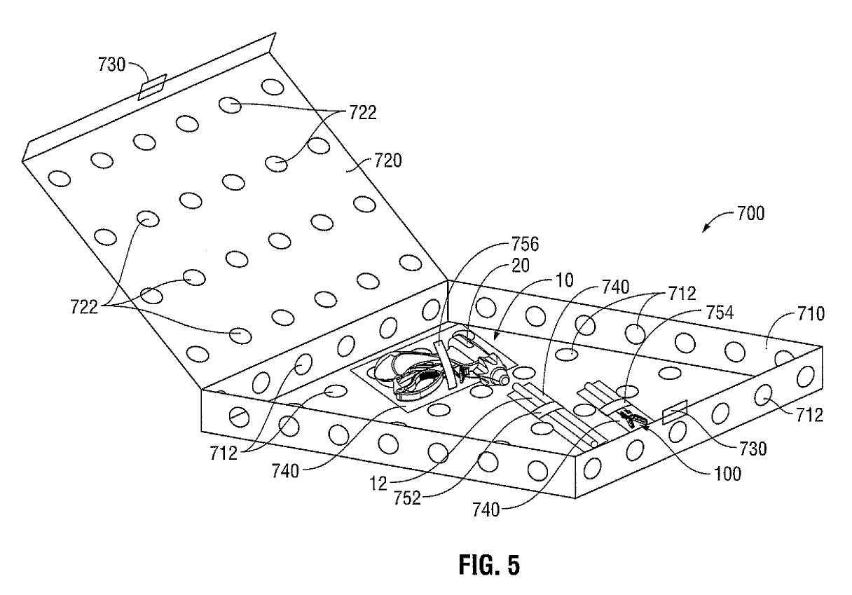

As best shown in FIG. 3, sterilization case 500 includes one or more releasable locking fixtures 552, 554, 556 configured to secure forceps 10 within base 510 in fixed position relative thereto. More specifically, sterilization case 500 includes a pair of fixtures 552, 554 engaged to base 510 and configured for securing shaft 12 of forceps 10 thereto, and a third fixture 556 engaged to base 510 and configured for securing housing 20 of forceps 10 thereto. However, greater or fewer than three fixtures 552, 554, 556 may be provided and/or fixtures 552, 554, 556 may be configured to secure various different surgical instrument within base 510 of sterilization case 500. Locking fixtures 552, 554, 556 may include straps, latches, buckles, or any other suitable releasably engagable mechanisms. Further, it is envisioned that locking fixtures 552, 554, 556 be extendable, or adjustable to facilitate securing various different surgical instruments within base 510 and/or that locking fixtures 552, 554, 556 be configured such that they do not substantially interfere with the sterilization of the portions of forceps 10 disposed adjacent to fixtures 552, 554, 556. As will be described below with reference to sterilization case 700 (FIG. 5), forceps 10 may be disassembled into several components, e.g., shaft 12, housing 20 and end effector assembly 100 may be disengaged from one another, to facilitate sterilization of forceps 10. Such a feature may apply similarly to sterilization case 500, e.g., each of the components of forceps 10 may be secured within sterilization case 500 via one or more of locking fixtures 552, 554, 556.

Referring now to FIGS. 1-3, in use, when it is desired to sterilize forceps 10, e.g., after grasping, sealing and/or dividing tissue, as described above, or after performing any other surgical task, electrosurgical cable 310 is disconnected from forceps 10. Forceps 10 is then positioned within base 510 and is secured therein via locking fixtures 552, 554, 556. With forceps secured within base 510, cover 520 may be secured about base 510, e.g., via latching features 530, to enclose forceps 10 therein. Thereafter, cable 320 is coupled to one of the inputs 410 of generator 400 at one of the ends thereof and to input 540 of sterilization case 500 at the other end thereof. Alternatively, any other suitable sterilization power source (not shown) may be coupled to sterilization case 500.

With cable 320 coupling generator 400 and sterilization case 500 to one another, generator 400 may be activated, e.g., via user interfaces 420, to transmit energy into sterilization case 500. For example, relatively high voltage energy, e.g., RF energy, may be supplied to sterilization case 500 to ionize the air (fluid) surrounding forceps 10, thus creating a corona about forceps 10 to sterilize forceps 10 via corona discharge. Either positive or negative corona discharge may be used to sterilize forceps 10. Further, cable 320 may include a feed for supplying a gas or fluid to sterilization case 500 to facilitate corona discharge (or, alternatively, a separate cable (not shown) may be provided for supplying gas or fluid to sterilization case 500). In such an embodiment, generator 400 may include or may be coupled to a gas or fluid source (not explicitly shown) for providing gas/fluid through cable 320 to sterilization case 500 to facilitate sterilization of forceps 10.

Alternatively, forceps 10 (or any other electrosurgical instrument to be sterilized) may be formed from transparent components, or materials. In such an embodiment, generator 400 is activated to supply UV energy to sterilization case 500. The transparent components of forceps 10 absorb the UV energy and are thereby sterilized.

In other embodiments, generator 400 is configured to supply gamma ray energy to sterilization case 500 in order to sterilize forceps 10. In such an embodiment, sterilization case 500 includes, or is formed from a shielding material that inhibits the passage of gamma rays therethrough to protect the user from exposure to radiation. Other suitable sterilization techniques for sterilizing a surgical instrument, e.g., forceps 10, via applying electromagnetic energy thereto are also contemplated.

Turning now to FIG. 4, another embodiment of a sterilization case 600 is shown. Sterilization case 600 is configured to secure forceps 10 therein and is similar to sterilization case 500, except that sterilization case 600 includes an input 610 that is configured to couple directly to one of the inputs 410 of generator 400, i.e., without the use of cable 320 (FIGS. 2-3). As such, sterilization case 600 may be latched, or secured to generator 400, e.g., via latches 620, while inputs 410, 610 of generator 400 and sterilization case 600, respectively, are electrically coupled to one another. Generator 400 may then be operated as described above to sterilize forceps 10 according to any of the sterilization techniques discussed above, or in accordance with any other suitable sterilization technique.

Referring now to FIG. 5, another embodiment of a sterilization case is shown generally identified by reference numeral 700. Sterilization case 700 is formed from any suitable material configured to withstand sterilization equipment, e.g., an autoclave (not shown) or other such equipment). Sterilization case 700 generally includes a base 710 and a cover 720 hingably couped to base 710 and movable relative thereto between an open position, for inserting and removing forceps 10 therefrom, and a closed position, wherein cover 720 is latched to base 710 via complementary latching components 730 to enclose forceps 10 therein. Both base 710 and cover 720 include a plurality of apertures 712, 722, respectively, defined therethrough to permit sterilization of a surgical instrument, e.g., forceps 10, disposed therein, as will be described in greater detail below. Base 710 and/or cover 720 may also include sterilization instructions 515 (FIG. 2) disposed thereon.

Similar to sterilization case 500 (FIGS. 2-3), sterilization case 700 is configured to retain forceps 10 therein. More specifically, sterilization case 700 includes a first fixture 752 engaged to base 710 and configured to retain shaft 12 of forceps 10 in position relative to base 710, a second fixture 754 engaged to base 710 and configured to retain end effector assembly 100 in position relative to base 710, and a third fixture 556 engaged to base 710 and configured to secure housing 20 of forceps 10 to base 710. As shown in FIG. 5, forceps 10 is disassembled into three components, shaft 12, end effector assembly 100, and housing 20. However, forceps 10 (or any other surgical instrument used in conjunction with sterilization case 700) may be separable into greater or fewer than three components and, accordingly, sterilization case 700 may include greater or fewer than three fixtures 752, 754, 756 to secure the components within sterilization case 700. Fixtures 752, 754, 756, similarly as described above, may include straps, latches, buckles, or any other suitable releasably engagable mechanisms, and/or may be adjustable to accommodate various different surgical instruments within base 710. Further, fixtures 752, 754, 756 (or any other suitable fixture provided) are configured such that they do not substantially interfere with the sterilization of the portions of forceps 10 disposed adjacent to fixtures 752, 754, 756. For example, fixtures 752, 754, 756 may include a plurality of apertures or slots (not explicitly shown) defined therethrough to provide greater access to the portions of forceps 10 disposed under, or in close proximity to fixtures 752, 754, 756, thus facilitating sterilization of these portions.

As can be appreciated, the ability to secure all the components of the surgical instrument within base 710 of sterilization case 700 helps ensure that all components are sterilized and also inhibits misplacement of one of more components thereof. To this end, markings 740, e.g., component terms or identifiers, pictures, outlines, symbols, etc. may be disposed on base 710 adjacent to or surrounding each of fixtures 752, 754, 756 to indicate to the user where to secure each of the components of the surgical instrument to sterilization case 700. Such a feature ensures that each of the components of the surgical instrument are maintained together by allowing the user to quickly ascertain if any components are missing or are improperly secured, and/or allows the user to ascertain if any additional components that do not belong, e.g., disposable components, components of other instrumentation, are present.

With the components of forceps 10 to be sterilized secured within sterilization case 700 (and without and additional, unwanted components disposed therein), cover 720 may be latched to base 710 via latching components 730 to enclose forceps 10 therein. Thereafter, sterilization case 700 may be placed in an appropriate sterilization device, e.g., in accordance with the instructions 515 (FIG. 2) disposed thereon, to sterilize forceps 10 in preparation for re-use. For example, sterilization case 700 may be placed in an autoclave (not shown) for sterilization.

Forceps 10 (or any other suitable surgical instrument) and/or sterilization cases 500, 600, 700 may additionally or alternatively be configured for use with various other sterilization techniques including high-temperature sterilization, steam sterilization, low-temperature hydrogen peroxide sterilization (e.g., Sterrad or Steris sterilization), and/or EtO sterilization. Further, forceps 10 may be formed at least partially from a microbial resistive plastic or may be coated with Teflon to facilitate sterilization thereof.

From the foregoing and with reference to the various figure drawings, those skilled in the art will appreciate that certain modifications can also be made to the present disclosure without departing from the scope of the same. While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A surgical system, comprising:
a surgical instrument including an end effector assembly adapted to connect to a source of energy;
a generator configured to couple to the surgical instrument to supply energy to the end effector assembly of the surgical instrument; and
a sterilization case, the sterilization case configured to retain the surgical instrument therein and configured to couple to the generator, the generator configured to supply energy to the sterilization case when coupled thereto to sterilize the surgical instrument disposed therein.

2. The surgical system according to claim 1, wherein the sterilization case includes at least one fixture for securing the surgical instrument in position therein.

3. The surgical system according to claim 1, wherein the generator is configured to supply energy to the sterilization case to create a corona about the surgical instrument to sterilize the surgical instrument via corona discharge.

4. The surgical system according to claim 1, further comprising an electrosurgical cable, the electrosurgical cable configured to couple the generator to the surgical instrument.

5. The surgical system according to claim 4, wherein the electrosurgical cable is configured to couple the generator to the sterilization case.

6. The surgical system according to claim 1, wherein the sterilization case is configured to couple directly to the generator.

7. The surgical system according to claim 1, wherein the sterilization case includes instructions disposed on a surface thereof.

8. The surgical system according to claim 1, wherein the generator is configured to supply UV energy to the sterilization case to sterilize the surgical instrument.

9. The surgical system according to claim 8, wherein the surgical instrument is formed from transparent components to facilitate UV sterilization thereof.

10. The surgical system according to claim 1, wherein the generator is configured to supply gamma ray energy to the sterilization case to sterilize the surgical instrument.

11. A method of surgery, comprising the steps of:
providing a surgical instrument including an end effector assembly, a generator, and a sterilization case;
coupling the surgical instrument to the generator;
performing a surgical task wherein energy is supplied from the generator to the end effector assembly of the surgical instrument;
decoupling the surgical instrument from the generator;
positioning the surgical instrument within the sterilization case;
coupling the sterilization case to the generator; and
operating the generator to sterilize the surgical instrument disposed within the sterilization case.

12. The method according to claim 11, wherein the generator is configured to supply energy to the sterilization case to create a corona about the surgical instrument to sterilize the surgical instrument via corona discharge.

13. The method according to claim 11, wherein the sterilization case includes at least one fixture for securing the surgical instrument in position therein.

14. The method according to claim 11, further comprising the step of separating the surgical instrument into multiple components prior to positioning the surgical instrument in the sterilization case, to facilitate sterilization of the surgical instrument.

15. The method according to claim 11, wherein the generator is configured to supply UV energy to the sterilization case to sterilize the surgical instrument.

16. The surgical system according to claim 11, wherein the generator is configured to supply gamma ray energy to the sterilization case to sterilize the surgical instrument.

17. A sterilization case for a surgical instrument configured to be disassembled into a plurality of sterilizable components, the sterilization case comprising:
a base configured to receive each of the components of the surgical instrument therein;
a cover coupled to the base and moveable relative thereto between an open position permitting insertion and removal of the surgical instrument and a closed position wherein the surgical instrument is enclosed within the sterilization case; and
a plurality of fixtures disposed on the base and configured to releasably secure each of the sterilizable components of the surgical instrument to the base, each of the fixtures designated for releasably securing a particular component of the surgical instrument to the base such that all of and only the sterilizable components of the surgical instrument are securable within the base for sterilization thereof.

18. The sterilization case according to claim 17, further comprising a marking associated with each of the fixtures, each marking indicating which sterilizable component of the surgical instrument is to be secured to the fixture associated therewith.

19. The sterilization case according to claim 17, wherein at least one of the fixtures includes a plurality of apertures defined therethrough to provide access to a portion of the surgical instrument disposed in close proximity to the fixture to facilitate sterilization of that portion of the surgical instrument.

20. The sterilization case according to claim 17, wherein each of the base and cover includes a plurality of apertures defined therethrough to facilitate sterilization of the surgical instrument.
